(19) United States
(12) Patent Application Publication
(10) Pub. No.: US 2004/0082940 A1
(43) Pub. Date: Apr. 29, 2004

(54) Dermatological apparatus and method

(76) Inventors: Michael Black, Foster City, CA (US); David Eimerl, Livermore, CA (US)

Correspondence Address: COOLEY GODWARD LLP, ATTN: PATENT GROUP, 11951 FREEDOM DRIVE, SUITE 1700, ONE FREEDOM SQUARE- RESTON TOWN CENTER, RESTON, VA, 20190-5061, US

(21) Appl. No.: 10/278,582

(22) Filed: Oct. 23, 2002

(51) Int. Cl. A61B 18/20

(52) U.S. Cl. 606/9; 606/10

(57) ABSTRACT

A dermatological laser apparatus in accordance with the present invention may comprise a plurality of laser light sources, a corresponding plurality of optical delivery pathways, and a focusing system. The dermatological laser apparatus may also comprise a control system for controlling the operation of the plurality of laser light sources to generate a broad range of therapeutic treatment patterns on or within a layer of skin.

### PRIORITY

The present application claims priority from, and is a continuation-in-part of, commonly owned and assigned U.S. patent application Ser. No. 10/017,287, entitled “Multiple Laser Treatment” (prior Attorney Docket No. RLT-111/US), and U.S. patent application Ser. No. 10/020,270 entitled “Multiple Laser Diagnostics” (prior Attorney Docket No. RLT-112), which are incorporated herein by reference.

### FIELD OF THE INVENTION

This invention relates generally to laser systems. More particularly, the present invention relates to devices and methods for treating unwanted dermatological conditions.

### BACKGROUND OF THE INVENTION

Lasers have many useful applications to the treatment of surfaces. For example, laser heat-treating of metals has become a valuable industrial process, because it provides a way for selectively hardening specific areas of a metal part. Lasers have also become valuable medical instruments to treat various kinds of unwanted dermatological conditions (For an overview, refer to, for instance, a book edited by M. P. Goldman and R. E. Fitzpatrick entitled “Cutaneous Laser Surgery” and published in 1999 by Mosby; or a book edited by R. E. Fitzpatrick and M. P. Goldman entitled “Cosmetic Laser Surgery” and published in 2000 by Mosby). Current medical laser devices and methods include a laser system to generate a specific wavelength tailored to a particular dermatological application (See, for instance, U.S. Pat. No. 5,336,217 to Buys; U.S. Pat. No. 5,964,749 to Eckhouse; U.S. Pat. No. 6,120,497 to Anderson; or U.S. Pat. No. 6,273,885 to Koop).

Even though, the current devices and methods may work well for their intended purposes, they pose several drawbacks. For instance, with today's demand and wide variety of different dermatological applications, there is a strong desire to develop more versatile devices that can handle various kinds of dermatological applications rather than a single device tailored for a particular application. Furthermore, laser treatment, in particular if the targeted tissue is subcutaneous, may develop unwanted damage of non-targeted tissue (For an overview of laser-tissue interaction, refer to, for instance, the paper by R. R. Anderson and E. V. Ross in a paper entitled “Laser-Tissue Interactions” in the book edited by R. E. Fitzpatrick and M. P. Goldman entitled “Cosmetic Laser Surgery” and published in 2000 by Mosby, pp. 1-30). Some of the current devices and methods have attempted to overcome this negative effect by including a cooling device to cool down the non-targeted tissue (usually the skin) and thereby minimize the heat development and damage to that tissue (See, for instance, U.S. Pat. No. 5,964,749 to Eckhouse; U.S. Pat. No. 6,120,497 to Anderson; or U.S. Pat. No. 6,273,885 to Koop). However, such cooling devices add complexity to the device and also do not necessarily guarantee the anticipated cooling and damage reduction of non-targeted tissue, because the amount of cooling and the effect of the cooling device are unknown. Yet another drawback of current devices arises from the fact that a clinician typically places and holds the device in proximity or close to the skin during the treatment. This might work well for a single treatment, however, if any follow-up treatment is required, it might be difficult, if not impossible, to place and hold the device at the same place and aim the light beam at the same target area. Furthermore, the current devices or methods often lack accuracy in applying the dermatological treatment and do not provide any feedback to a clinician over the efficacy of an applied dermatological treatment.

Accordingly, there is a need to develop new dermatological devices and methods that provide versatility and flexibility. There is a further need to develop devices and methods that are not dependent on coolant devices to minimize tissue damage. There is yet another need to develop devices and methods that provide for better accuracy of the applied treatment. There is still another need to develop devices and methods that enable a clinician to obtain feedback concerning the efficacy the applied treatment.

### SUMMARY OF THE INVENTION

In one particularly innovative aspect, the present invention is directed to a dermatological laser apparatus that may be used to treat a wide variety of diseases, disorders, and conditions associated with the skin. In one preferred embodiment, a dermatological laser apparatus in accordance with the present invention may comprise a plurality of laser light sources, a corresponding plurality of optical pathways, and a focusing system for focusing energy generated by the respective laser light sources and delivered by the corresponding optical pathways upon an area of tissue on the surface of, or within, the skin of a patient.

In another particularly innovative aspect, a dermatological laser system in accordance with the present invention may be used to treat tissue using a pattern of beams that may vary in frequency, intensity, duration, focus depth, or the like to deliver a precise treatment pattern that is designed to address a particular dermatological condition while minimizing or reducing heating of adjacent or surrounding tissues. In this regard, it may be particularly advantageous to generate therapeutic patterns employing microscopic beam spot sizes when treating a particular area of tissue.

In still other innovative aspects, the present invention contemplates the use of an optical focusing system and/or vacuum assembly to deform an area of skin during treatment. In this fashion, the focusing system can more accurately focus energy delivered by the various optical pathways upon a targeted area of tissue to be treated.

### BRIEF DESCRIPTION OF THE DRAWINGS

The objectives and advantages of the present invention will be understood by reading the following detailed description in conjunction with the drawings, in which:

FIG. 1 is a block diagram of a dermatological laser system in accordance with a first embodiment of the present invention;

FIG. 2 is a block diagram of a dermatological laser system in accordance with a second embodiment of the present invention;

FIG. 3 illustrates how a plurality of laser light sources and optical pathways may be arranged and distributed within an array in accordance with various aspects of the present invention;

FIG. 4 illustrates how an array in accordance with various aspects of the present invention may be used to generate unique therapeutic patterns;

FIG. 5 illustrates several exemplary therapeutic treatment patterns that may be applied to an area of human skin;

FIG. 6 illustrates how a focusing lens may be employed within an embodiment of the present invention;

FIG. 7 illustrates how a focusing lens may be employed to function as a skin deformation apparatus within another embodiment of the present invention;

FIG. 8 illustrates how a lens or focusing system may be used to stretch an area of skin in accordance with an embodiment of the present invention;

FIG. 9 is a block diagram of a vacuum system that may be used for skin deformation in accordance with an embodiment of the present invention;

FIG. 10 is a block diagram illustrating a top view of a dermatological device that incorporates a target tissue viewing system in accordance with an embodiment of the present invention; and

FIG. 11 is a block diagram illustrating a recording and display system in accordance with an embodiment of the present invention.

### DETAILED DESCRIPTION

Although the following detailed description contains many specifics for the purposes of illustration, anyone of ordinary skill in the art will readily appreciate that many variations and alterations to the following exemplary details are within the scope of the invention. Accordingly, the following preferred embodiment of the invention is set forth without any loss of generality to, and without imposing limitations upon, the claimed invention.

The present invention provides an advanced dermatological laser apparatus and method that can be used with great flexibility and versatility to treat a wide variety of unwanted dermatological conditions such as, but not limited to, cosmetic laser applications, skin rejuvenation, laser hair or tattoo removal, and other medical laser treatments. Examples of these applications are the treatment of wrinkles, leg veins, acne scars, birthmarks, or port wine stains. However, as a person of average skill in the art would readily appreciate the present invention could be used for any type of dermatological treatment. For an overview of possible applications related to of the present invention, one is referred to, for instance, a book edited by M. P. Goldman and R. E. Fitzpatrick entitled “Cutaneous Laser Surgery” and published in 1999 by Mosby; or a book edited by R. E. Fitzpatrick and M. P. Goldman entitled “Cosmetic Laser Surgery” and published in 2000 by Mosby.

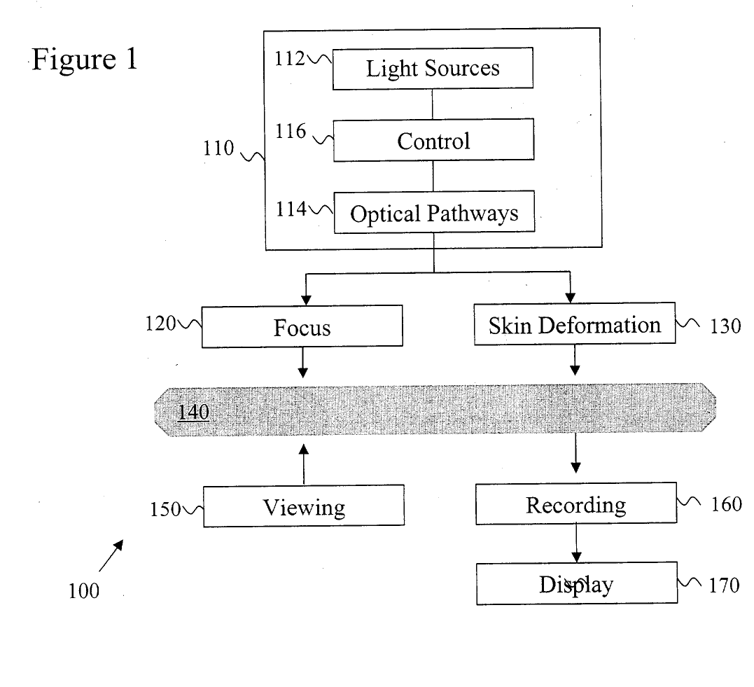

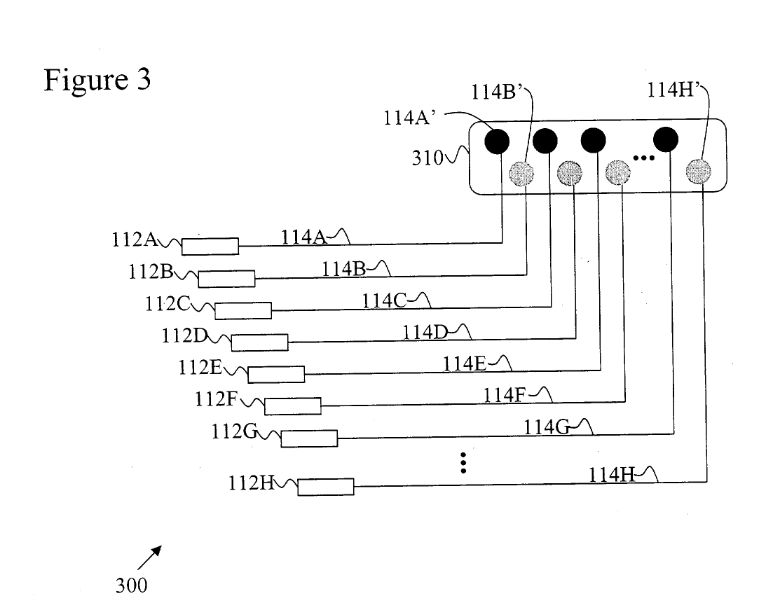

FIG. 1 shows a dermatological laser apparatus 100 in accordance with a first embodiment of the present invention. Dermatological laser apparatus 100 includes an optical delivery system 110, which includes a plurality of laser light sources 112 and optical pathways 114. The laser light sources 112 in optical delivery system 110 preferably are connected, on a one-by-one basis, to optical pathways 114, as illustrated in FIG. 3. The idea here is that each laser light source 112A-112H, is capable of delivering a light beam through it own optical pathway 114A-114H connection, in optical pathways 114, to a targeted portion of a human skin 140. Those skilled in the art will appreciate, however, that the optical delivery system 10 may include other optical elements, such as lens systems or waveguides (not shown) to deliver the beams generated by the plurality of laser light sources 112 to an area of tissue to be treated, and that the present invention is not limited to the number of light sources 112 illustrated herein, which could be any number from two light sources on up. Laser light sources 112 can be any type of light source that is capable of delivering a wavelength ranging from roughly 400 nm to 5 μm; i.e. a wavelength range that covers a wide variety of dermatological effects (See, for instance, the book edited by R. E. Fitzpatrick and M. P. Goldman entitled “Cosmetic Laser Surgery” and published in 2000 by Mosby). Exemplary laser light sources 112 include diode lasers, Nd:YAG lasers, argon-ion lasers, He—Ne lasers, carbon dioxide lasers, eximer lasers, ruby lasers, and the like. However, the selection of the type of laser light source 112 in optical delivery system 110 is dependent on the range of dermatological applications that one would like to cover using the apparatus 100. Optical delivery system 110 may include just one particular kind of light source capable of delivering one wavelength or a wavelength range. However, optical delivery system 110 may also include a mixture of two or more different types of light sources. Preferably, optical delivery system 110 includes a mixture of different light sources 112 that are capable of delivering a variety of different wavelengths ranging from 400 nm to 5 μm. Light sources 112 are preferably diode lasers. Since the optical delivery system 110 has the option of providing a variety of different light sources 112 that are connected, on a one-by-one basis, to optical pathways 114, a pattern of light beams can be created and delivered to a targeted portion of a human skin 140. To accomplish such a pattern, apparatus 100 preferably includes a control system 116 to select and control the light source parameters of each light source 112A-112H in light sources 112 (e.g. power, wavelength if a range can be selected in this particular light source) as well as the timing and duration for each light source 112 to deliver its light beam. Control system 116 may select and control one or more light beams in a pattern. The pattern can either be a randomized pattern or a programmed pattern. As a person of average skill in the art would readily appreciate, control system 116 preferably includes a computer interface to enable a user to change and/or program control system 116. Such a person also would readily recognize that the control system 116 may be electronically coupled directly or indirectly to the laser light sources 112 and may be implemented using (1) dedicated hardware or logic elements, implemented, for example, in a programmable gate array; (2) a typical microprocessor or central processing unit (CPU) available, for example, from Intel Corp.; or (3) any of a number of personal computer, web appliance, and personal digital assistant products that are now available on the market. As used herein, the term “control means” shall be construed to include any of the foregoing products and their equivalents.

FIG. 3 shows an example of light sources 112A-H connected through optical pathways 114A-H. As it is shown in FIG. 3, the ends 114A′-H′ of optical pathways 114A-H could be arranged and distributed in an array 310. Optical pathways 114A-H are preferably optical fibers with a diameter ranging from single mode fiber diameters to 1 mm. However, as a person of average skill in the art would readily appreciate, the optical pathways are not limited to optical fibers and, for example, could be any type of waveguide. Such a person also would appreciate that optical elements such as lens and mirror systems may be employed within the context of the present invention to provide the functionality of the optical pathways 114.

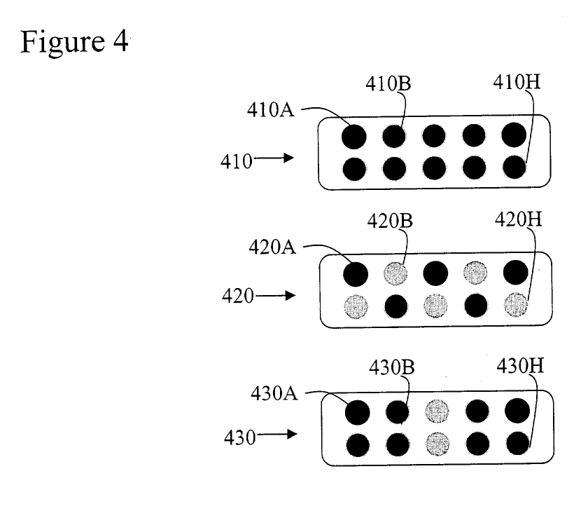

FIG. 4 shows examples of arrays 410-430 each with 10 optical pathway outputs 410A-H, 420A-H and 430A-H. In array 410, optical pathways 410A-H output the same parameters of light beams. However, in array 420 and 430, optical pathways 420A-H and 430A-H output different parameters of light beams as indicated by the black and gray circles, e.g. 420A and 420B respectively in array 420. A person of average skill in the art would readily appreciate that a variety of different parameters (wavelength, power, duration, frequency, etc.) can be selected and that the parameters are not limited to just two different parameters as illustrated by the black and gray circles.

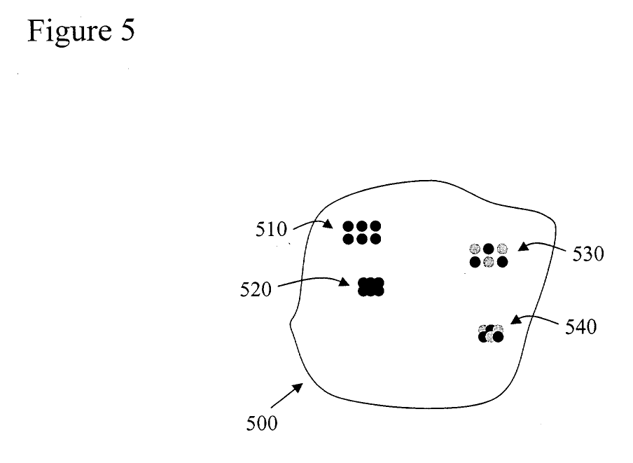

FIG. 5 shows a targeted portion of a human skin 500 with some exemplary patterns of light beams 510-540. Patterns 510 and 530 show a pattern where the light beams are distributed, whereas patterns 520 and 540 show overlap of the light beams. The pattern of light beams can be arranged with and/or without overlap. Such variations in patterns can be established electronically and/or mechanically by steering the optical pathways 114 to obtain the desired pattern. For instance, an optical pathway 114 could be rotated around its X, Y or Z axis or translated in its X, Y and Z direction. Not shown in FIGS. 3-5 are the timing aspects of the different light beams in each pattern. However, as one of average skill in the art would readily appreciate, some or all of the light beams can be controlled by control system 116 in terms of frequency, interval and duration, and can be combined in a variety of different ways with the other light beams.

Referring back to FIG. 1, apparatus 100 further includes a focusing system 120. Focusing system 120 preferably includes a spherical lens to focus the power of one or more light beams at a targeted portion of a human skin of tissue 140. Indeed, in a particularly preferred form of the present invention, it is desirable to focus one or more light beams at a microscopic area within a range up to about 1.5 mm below the surface of the skin. Moreover, because it is contemplated that a dermatological laser apparatus 100 in accordance with the present invention may be used to treat a wide variety of skin conditions, and conditions associated with related biologic structures, those skilled in the art will recognize that the focusing system 120 may be used to focus a beam upon virtually any area or structure within the epi-dermis, dermis, or hypo-dermis regions of the skin. Those skilled in the art will also appreciate that where it is desired to achieve very small or microscopic spot sizes or beam diameters, it may be useful to employ single mode optical fibers within the optical pathways 114.

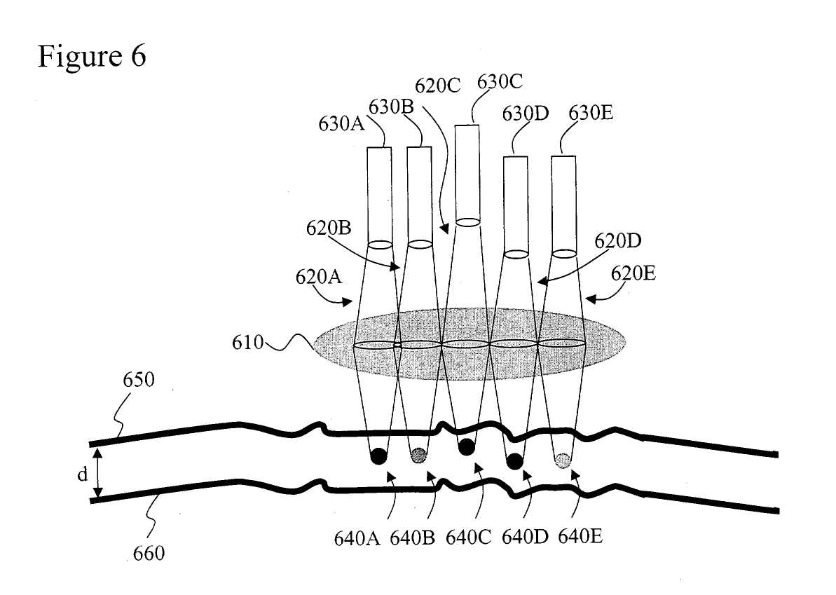

As it is shown in FIG. 6, focusing system 610 preferably focuses the power of light beams 620A-E that originate form optical pathways 630A-E, respectively, to spots 640A-E up to 1.5 mm (distance d measures the distance between human skin 650 and the bottom 660 of tissue 1.5 mm under human skin 650) underneath the targeted portion of human skin 650. Focusing system 610 can be placed anywhere between the optical pathways 114 and the skin. Focusing system 610 could also be adjusted to any position anywhere in between the optical pathways and the skin using, for instance, an electrical motor or any other device that is known in the art to position optical elements. FIG. 6 shows focusing system 610 as one lens, however, focusing system 610 is not limited to embodiments including a single lens and may also include to two or more lenses. Different lens sizes may be used ranging, for example, from a 2-mm diameter to a 2-inch diameter lens. Furthermore, focusing system 610 could be extended (not shown) with individual optical elements for each of the optical pathways 114. As indicated above, optical pathways 114 could be arranged and distributed differently. As is shown in FIG. 6, optical pathways 630-A-E are positioned at different positions relative to skin 650. One objective behind focusing system 120 is to focus the power of the light beams at the desired targeted area or spots, thereby minimizing damage as a result of overheating of tissue that needed to be penetrated to get to the desired target and/or tissue surrounding the desired target. As used herein, the term “focusing means” shall be construed to include any of the above-described lenses, lens systems, and optical elements together with all known equivalents to those structures.

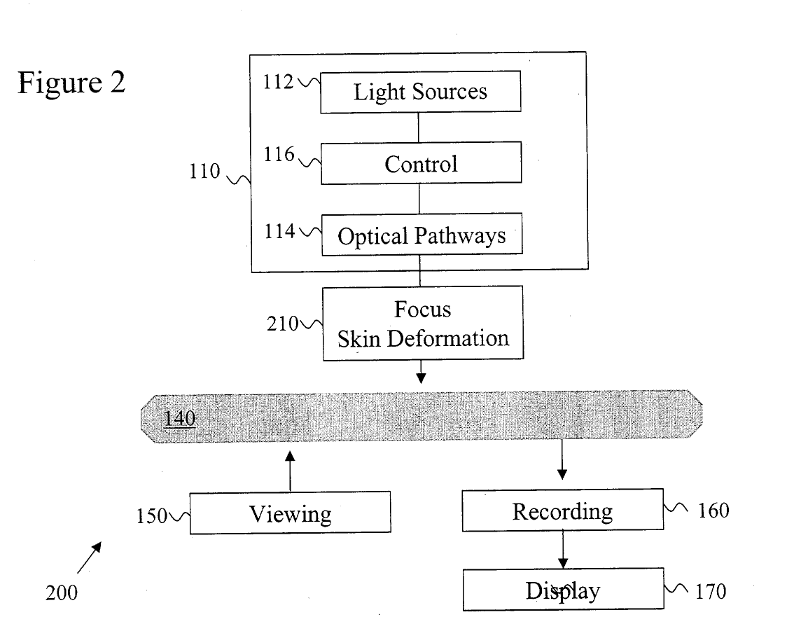

Referring back to FIG. 1, apparatus 100 also preferably includes a skin deformation system 130 to deform the targeted portion of a human skin 140. A primary objective of the skin deformation system 130 is to deform the skin in either a substantially flat manner or substantially concave manner. Subsequently, the subcutaneous tissue will also be deformed in a substantially similar manner as the skin. Skin deformation system 130 then provides a smoother working and treatment surface and allows for better accuracy and control over the delivery of the light beams. The present invention preferably employs two different kinds of skin deformation systems, which can either be used separate or in combination with each other. The first type of skin deformation system 130 uses stretching by pressing the focusing system 116 against the skin, whereas the second type of skin deformation system 130 uses stretching by applying suction to the skin. As is shown in FIGS. 1 and 2, focusing system 120 and skin deformation system 130 could be separate or could be combined as shown by focusing/skin deformation system 210 in apparatus 200.

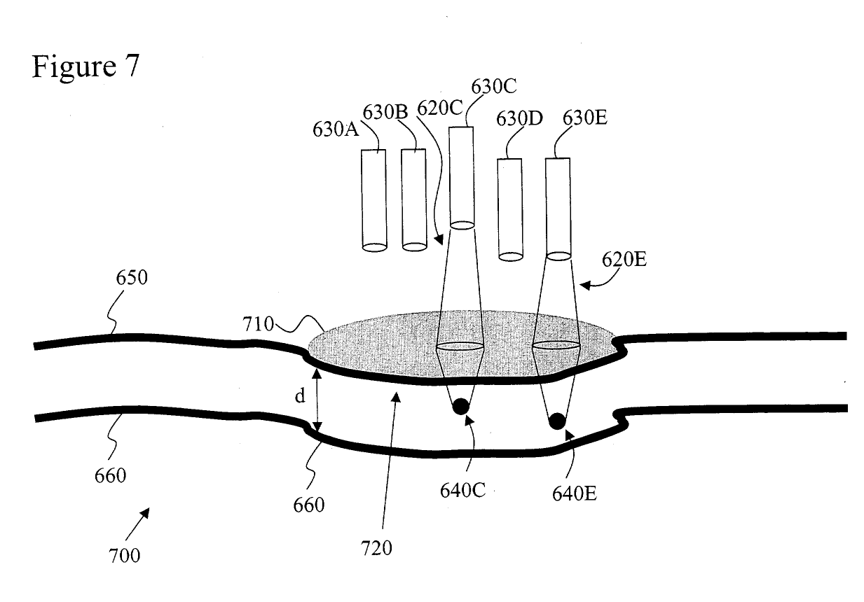

In one particular embodiment 700 of the present invention, skin deformation is taught as the stretching of a skin area 720 by using focusing system 710 and applying it to skin area 720. Since focusing system 710 is already an integral part of the dermatological laser apparatus 700 of the present invention, it would reduce the number of parts in the dermatological apparatus 700 to use focusing system 710 for focusing as well as for skin deformation. As it is shown in FIG. 7, the focusing system 710 comprises a lens that is placed against skin area 720 and as a result skin area 720 stretches in a more or less uniform surface. As mentioned above, the position of the optical pathways can be adjusted and by having this more or less uniform surface, the light beams can be more precisely applied and focused at the desired spots.

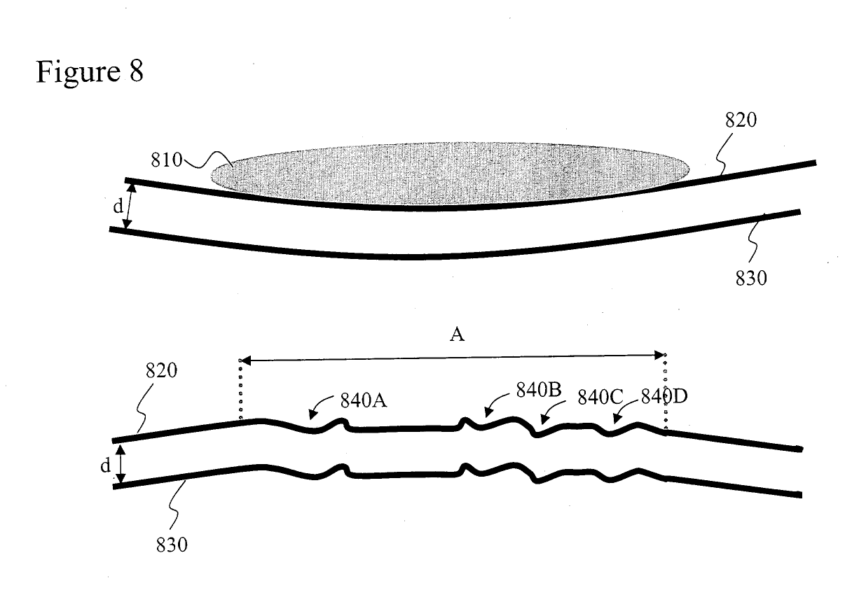

FIG. 8 shows another embodiment in which focusing system 810 is used to stretch an area R of skin 820. In this particular example, the dermatological condition involves wrinkles 840A-D. Due to the application of focusing system 810 to area R of skin 820, area R is stretched and consequently wrinkles 840A-D are stretched. Furthermore, the subcutaneous tissue, indicated by bottom layer 830 and depth d, is stretched to a substantially similar extent as skin 820.

As mentioned above, the second type of skin deformation system 910, which may be used in accordance with preferred embodiments of the present invention, achieves tissue stretching by applying suction to an area R of skin 820.

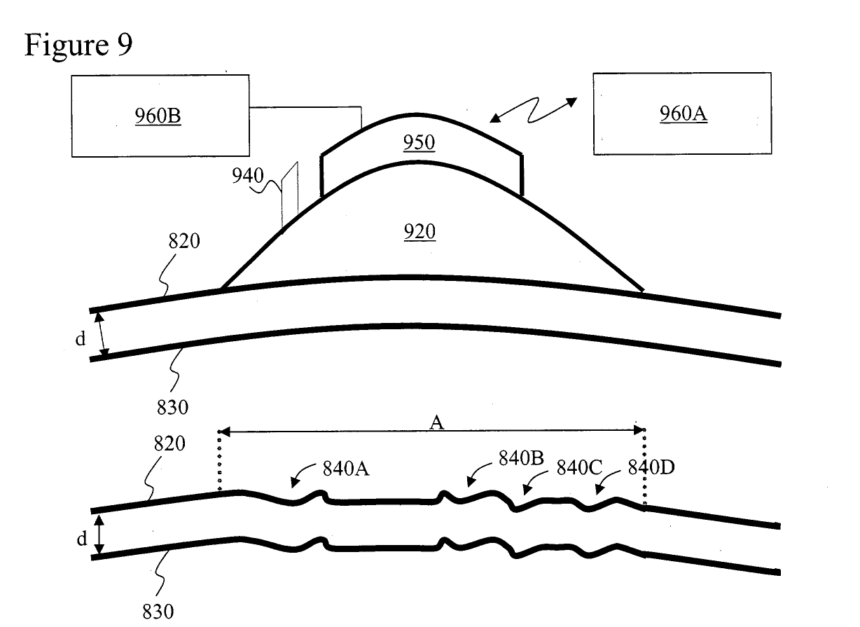

FIG. 9 shows skin deformation system 910 as a vacuum system. Vacuum system 910 may include a cup 920 that is placed at the skin 930. Cup 920 could take any type of shape as long as it provides an airtight seal with skin 930. Cup 920 includes an adapter 940 that enables one to suck out the air from the area inside cup 920 and skin 930. As a person of average skill would readily appreciate, vacuum system 910 may further include a control system (not shown) for adjusting the vacuum to create an appropriate and desired deformation of skin 930. In the particular example, the optical delivery system 950 may be attached to the top of cup 920. For instance, light sources 112, control system 116, and optical pathways 114 (shown in FIG. 1) may be placed on top of cup 920. However, some part of the control system 116 also may be placed remotely using a wireless connection 960A or via a tether 960B. In this particular example, the dermatological condition also involves wrinkles 830A-D. Due to the vacuum applied to skin 930, skin 930 has taken a concave shape and consequently wrinkles 830A-D have been stretched. Furthermore, the subcutaneous tissue, indicated by bottom layer 840 and depth d, has become concave to a substantially similar extent as skin 820. The term “skin deformation means” shall be construed herein to cover any of the above-described structures for stretching an area of human skin together with all known equivalents to those structures.

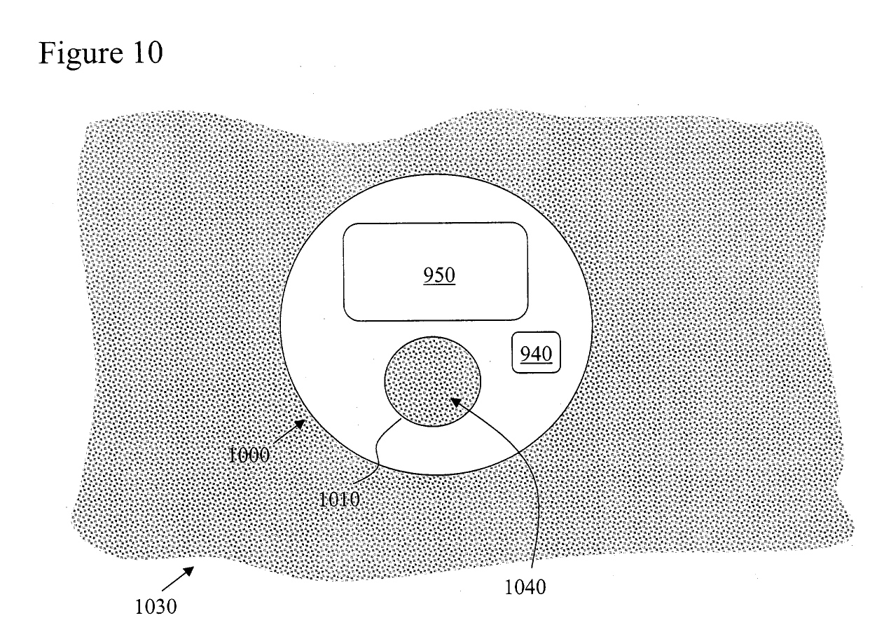

Referring back to FIGS. 1 and 2, the dermatological laser apparatus 100 and 200 may further include a viewing system 150, a recording system 160, and a display system 170. Viewing system 150 enables a user to view the targeted portion 1040 of the human skin 1030. FIG. 10 shows a top view of dermatological apparatus 1000 with a viewing system 1010 which could, for instance, be a circular area of transparent material (not shown) so that the user can view the targeted area of skin 1030. The circular area could be inserted in the cup as described above. Viewing system 160 also may include a coating to protect the user's eyes from reflections of the light beams. Viewing system 160 may also be as simple as an opening without any transparent material. In this particular case, the user should wear protective eye-apparels. The present invention may also include a system to dispose a chemical agent on the skin to make the skin more or less transparent. This would improve the view to the user of the targeted portion 1040 of the human skin 1030.

Recording system 160 preferably has the ability to record any of the reflected light and may, for instance, comprise an infrared camera or CCD device to record reflections from the light beams in the infrared spectrum or a visible camera or CCD device to record reflections from the light beams in the visible spectrum. Various kinds of recording devices and techniques can be used, as they are well known in the art.

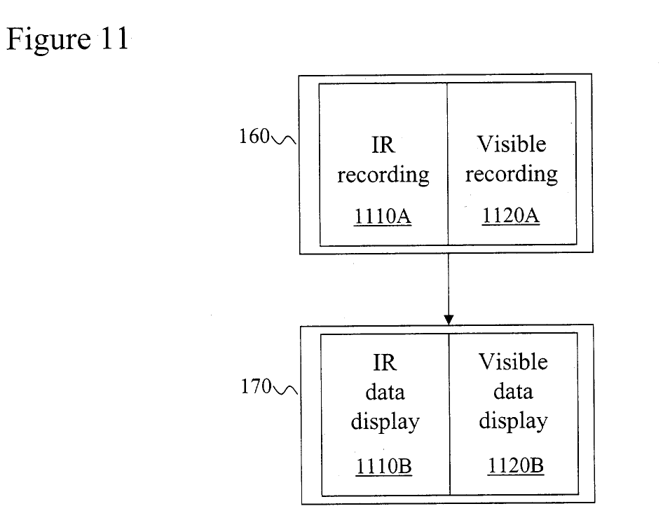

As is shown in FIG. 11, once infrared or visible reflections are recorded 1110A, 1120A, the recorded reflections or radiation can then be displayed as infrared data 1110B or visible data 1120B, respectively, using any kind of displaying system 1120. Examples of the display system include, for example, a computer screen, flat panel display, personal digital assistant, wireless communication devices that allows display of data, or the like. Display system also preferably has the ability to process some of the recorded data using a computer device or an integrated circuit. For instance, different parameters could be calculated or determined such as, but not limited to, the temperature of the skin or targeted areas, and the area of skin that has been treated.

The present invention has now been described in accordance with several exemplary embodiments, which are intended to be illustrative in all aspects, rather than restrictive. Thus, the present invention is capable of many variations in detailed implementation, which may be derived from the description contained herein by a person of ordinary skill in the art. All such variations are considered to be within the scope and spirit of the present invention as defined by the following claims and their legal equivalents.

What is claimed is:

1. A dermatological laser apparatus, comprising:
a plurality of light source and optical pathway connections, wherein each light source in said plurality of light source and optical pathway connections is capable of delivering a light beam through its connected optical pathway to a targeted portion of a human skin;
a control means to select and control said light sources to deliver one or more light beams in a pattern; and
a focusing means to focus the power of said delivered light beams to tissue up to 1.5 mm underneath said targeted portion of said human skin.

2. The apparatus as set forth in claim 1, further comprising a skin deformation means to deform said targeted portion of said human skin in a substantially flat manner.

3. The apparatus as set forth in claim 1, further comprising a skin deformation means to deform said targeted portion of said human skin in a substantially concave manner.

4. The apparatus as set forth in claim 1, further comprising a skin deformation means to deform said targeted portion of said human skin wherein said skin deformation means comprises a vacuum means to apply a vacuum at said targeted portion of said human skin.

5. The apparatus as set forth in claim 4, wherein said vacuum means further comprises a means for adjusting said vacuum to create an appropriate skin deformation.

6. The apparatus as set forth in claim 1, further comprising a viewing means to enable a user to view said targeted portion of said human skin, wherein said viewing means comprises a coating to protect said user's eyes from reflections of said light beams.

7. The apparatus as set forth in claim 1, further comprising a means for adjusting the distance between said optical pathways and said targeted portion of said human skin.

8. The apparatus as set forth in claim 1, wherein said focusing means is a skin deformation means to deform said targeted portion of said human skin.

9. The apparatus as set forth in claim 1, wherein said focusing means is a spherical lens.

10. The apparatus as set forth in claim 1, wherein said focusing means comprises one or more focusing elements for each of said light source and optical pathway connections.

11. The apparatus as set forth in claim 1, wherein said light sources are diodes lasers.

12. The apparatus as set forth in claim 1, wherein said light sources having wavelengths ranging from 400 nm to 5 μm.

13. The apparatus as set forth in claim 1, wherein said light sources having wavelengths resulting in dermatological effects.

14. The apparatus as set forth in claim 1, wherein said pattern is a randomized pattern of said light beams.

15. The apparatus as set forth in claim 1, wherein said pattern is a programmed pattern of said light beams.

16. The apparatus as set forth in claim 1, wherein said pattern of said light beams comprises two or more different wavelengths.

17. The apparatus as set forth in claim 1, wherein said control means controls parameters of each of light source, wherein parameters are timing, duration or power of each of said light beams.

18. The apparatus as set forth in claim 1, wherein said optical pathway is an optical fiber.

19. The apparatus as set forth in claim 1, wherein said optical pathways are arranged and distributed to deliver focused light beams with partial overlap at said tissue underneath said targeted portion of said human skin, to deliver said focused laser beams without overlap at said tissue underneath said targeted portion of said human skin, or to deliver said some focused light beams without overlap and some focused light beams with overlap at said tissue underneath said targeted portion of said human skin.

20. The apparatus as set forth in claim 1, further comprising a recording means to record said reflected radiation from said targeted portion of said human skin.

21. The apparatus as set forth in claim 20, wherein said recording means is an infrared camera or a visible camera.

22. The apparatus as set forth in claim 20, further comprising a means for displaying data of said recorded radiation.

23. The apparatus as set forth in claim 1, further comprising means to dispose a chemical agent to make said skin more or less transparent.

24. A dermatological laser apparatus, comprising:
an optical delivery means, wherein said optical delivery means comprises an array of light source and optical pathways connections and wherein each light source in said optical delivery means is capable of delivering a light beam through its connected optical pathway to a targeted portion of a human skin, wherein said optical delivery means delivers one or more of said light beams in a pattern, wherein said one or more of said light sources comprises light beam parameters that results in dermatological effects; and
a focusing means, wherein said focusing means focuses the power of said delivered light beams to tissue up to 1.5 mm underneath said targeted portion of said human skin and wherein said focusing means deforms said targeted portion of said human skin.

25. The apparatus as set forth in claim 24, wherein said optical delivery means comprises a control means to select and control said light sources to deliver one or more light beams in said pattern.

26. The apparatus as set forth in claim 24, further comprising a skin deformation means to deform said targeted portion of said human skin wherein said skin deformation means comprises a vacuum means to apply a vacuum at said targeted portion of said human skin.

27. The apparatus as set forth in claim 26, wherein said vacuum means further comprises a means for adjusting said vacuum to create an appropriate skin deformation.

28. The apparatus as set forth in claim 24, further comprising a viewing means to enable a user to view said targeted portion of said human skin, wherein said viewing means comprises a coating to protect said user's eyes from reflections of said light beams.

29. The apparatus as set forth in claim 24, further comprising a means for adjusting the distance between said optical pathways and said targeted portion of said human skin.

30. The apparatus as set forth in claim 24, wherein said focusing means is a skin deformation means to deform said targeted portion of said human skin.

31. The apparatus as set forth in claim 24, wherein said focusing means comprises one or more focusing elements for each of said light source and optical pathway connections.

32. The apparatus as set forth in claim 24, further comprising a recording means to record said reflected radiation from said targeted portion of said human skin.

33. The apparatus as set forth in claim 32, wherein said recording means is an infrared camera or a visible camera.

34. The apparatus as set forth in claim 32, further comprising a means for displaying data of said recorded radiation.

35. The apparatus as set forth in claim 24, further comprising means to dispose a chemical agent to make said skin more or less transparent.

36. A dermatological laser apparatus, comprising:
an optical delivery means, wherein said optical delivery means comprises an array of light source and optical pathways connections and wherein each laser in said optical delivery means is capable of delivering a light beam through its connected optical pathway to a targeted portion of a human skin, wherein said optical delivery means delivers one or more of said light beams in a pattern, wherein said one or more of said light sources comprises light beam parameters that results in dermatological effects;
a skin deformation means to deform said targeted portion of said human skin, wherein said skin deformation means comprises a vacuum means to apply a vacuum at said targeted portion of said human skin; and
a focusing means, wherein said focusing means focuses the power of said delivered light beams to tissue up to 1.5 mm underneath said targeted portion of said human skin.

37. The apparatus as set forth in claim 36, wherein said optical delivery means comprises a control means to select and control said light sources to deliver one or more light beams in said pattern.

38. The apparatus as set forth in claim 36, wherein said vacuum means further comprises a means for adjusting said vacuum to create an appropriate skin deformation.

39. The apparatus as set forth in claim 36, further comprising a viewing means to enable a user to view said targeted portion of said human skin, wherein said viewing means comprises a coating to protect said user's eyes from reflections of said light beams.

40. The apparatus as set forth in claim 36, further comprising a means for adjusting the distance between said optical pathways and said targeted portion of said human skin.

41. The apparatus as set forth in claim 36, wherein said focusing means is used as a second skin deformation means to deform said targeted portion of said human skin.

42. The apparatus as set forth in claim 36, wherein said focusing means comprises one or more focusing elements for each of said light source and optical pathway connections.

43. The apparatus as set forth in claim 36, further comprising a recording means to record said reflected radiation from said targeted portion of said human skin.

44. The apparatus as set forth in claim 43, wherein said recording means is an infrared camera or a visible camera.

45. The apparatus as set forth in claim 43, further comprising a means for displaying data of said recorded radiation.

46. The apparatus as set forth in claim 36, further comprising means to dispose a chemical agent to make said skin more or less transparent.

47. A dermatological laser apparatus, comprising:
an optical delivery means, wherein said optical delivery means comprises an array of light source and optical pathways connections and wherein each laser in said optical delivery means is capable of delivering a light beam through its connected optical pathway to a targeted portion of a human skin, wherein said optical delivery means delivers one or more of said light beams in a pattern, wherein said one or more of said light sources comprises light beam parameters that results in dermatological effects;
a focusing means, wherein said focusing means focuses the power of said delivered light beams to tissue up to 1.5 mm underneath said targeted portion of said human skin, wherein said focusing means is also used as a skin deformation means to deform said targeted portion of said human skin.

48. The apparatus as set forth in claim 47, wherein said optical delivery means comprises a control means to select and control said light sources to deliver one or more light beams in said pattern.

49. The apparatus as set forth in claim 47, further comprising a second skin deformation means to deform said targeted portion of said human skin wherein said second skin deformation means comprises a vacuum means to apply a vacuum at said targeted portion of said human skin.

50. The apparatus as set forth in claim 49, wherein said vacuum means further comprises a means for adjusting said vacuum to create an appropriate skin deformation.

51. The apparatus as set forth in claim 47, further comprising a viewing means to enable a user to view said targeted portion of said human skin, wherein said viewing means comprises a coating to protect said user's eyes from reflections of said light beams.

52. The apparatus as set forth in claim 47, further comprising a means for adjusting the distance between said optical pathways and said targeted portion of said human skin.

53. The apparatus as set forth in claim 47, wherein said focusing means comprises one or more focusing elements for each of said light source and optical pathway connections.

54. The apparatus as set forth in claim 47, further comprising a recording means to record said reflected radiation from said targeted portion of said human skin.

55. The apparatus as set forth in claim 54, wherein said recording means is an infrared camera or a visible camera.

56. The apparatus as set forth in claim 54, further comprising a means for displaying data of said recorded radiation.

57. The apparatus as set forth in claim 47, further comprising means to dispose a chemical agent to make said skin more or less transparent.

58. A dermatological laser apparatus, comprising:
an optical delivery means, wherein said optical delivery means comprises an array of light source and optical pathways connections and wherein each light source in said optical delivery means is capable of delivering a light beam through its connected optical pathway to a targeted portion of a human skin, wherein said optical delivery means delivers one or more of said light beams in a pattern, wherein said one or more of said light sources comprises light beam parameters that results in dermatological effects;
(a) a skin deformation means to deform said targeted portion of said human skin;
a focusing means, wherein said focusing means focuses the power of said delivered light beams to tissue up to 1.5 mm underneath said targeted portion of said human skin; and
a recording means to record said reflected radiation from said targeted portion of said human skin.

59. The apparatus as set forth in claim 58, wherein said optical delivery means comprises a control means to select and control said light sources to deliver one or more light beams in said pattern.

60. The apparatus as set forth in claim 58, wherein said skin deformation means comprises a vacuum means to apply a vacuum at said targeted portion of said human skin.

61. The apparatus as set forth in claim 60, wherein said vacuum means further comprises a means for adjusting said vacuum to create an appropriate skin deformation.

62. The apparatus as set forth in claim 58, further comprising a viewing means to enable a user to view said targeted portion of said human skin, wherein said viewing means comprises a coating to protect said user's eyes from reflections of said light beams.

63. The apparatus as set forth in claim 58, further comprising a means for adjusting the distance between said optical pathways and said targeted portion of said human skin.

64. The apparatus as set forth in claim 58, wherein said focusing means is also used as said skin deformation means to deform said targeted portion of said human skin.

65. The apparatus as set forth in claim 58, wherein said focusing means comprises one or more focusing elements for each of said light source and optical pathway connections.

66. The apparatus as set forth in claim 58, wherein said recording means is an infrared camera or a visible camera.

67. The apparatus as set forth in claim 58, further comprising a means for displaying data of said recorded radiation.

68. The apparatus as set forth in claim 58, further comprising means to dispose a chemical agent to make said skin more or less transparent.

69. A dermatological laser apparatus, comprising:
an optical delivery means, wherein said optical delivery means comprises an array of light source and optical pathways connections and wherein each light source in said optical delivery means is capable of delivering a light beam through its connected optical pathway to a targeted portion of a human skin, wherein said optical delivery means delivers one or more of said light beams in a pattern, wherein said one or more of said light sources comprises light beam parameters that results in dermatological effects;
a skin deformation means to deform said targeted portion of said human skin;
a focusing means, wherein said focusing means focuses the power of said delivered light beams to tissue up to 1.5 mm underneath said targeted portion of said human skin; and
a viewing means to enable a user to view said targeted portion of said human skin, wherein said viewing means comprises a coating to protect said user's eyes from reflections of said light beams.

70. The apparatus as set forth in claim 69, wherein said optical delivery means comprises a control means to select and control said light sources to deliver one or more light beams in said pattern.

71. The apparatus as set forth in claim 69, wherein said skin deformation means comprises a vacuum means to apply a vacuum at said targeted portion of said human skin.

72. The apparatus as set forth in claim 71, wherein said vacuum means further comprises a means for adjusting said vacuum to create an appropriate skin deformation.

73. The apparatus as set forth in claim 69, further comprising a means for adjusting the distance between said optical pathways and said targeted portion of said human skin.

74. The apparatus as set forth in claim 69, wherein said focusing means is also used as said skin deformation means to deform said targeted portion of said human skin.

75. The apparatus as set forth in claim 69, wherein said focusing means comprises one or more focusing elements for each of said light source and optical pathway connections.

76. The apparatus as set forth in claim 69, further comprising a recording means to record said reflected radiation from said targeted portion of said human skin.

77. The apparatus as set forth in claim 76, wherein said recording means is an infrared camera or a visible camera.

78. The apparatus as set forth in claim 76, further comprising a means for displaying data of said recorded radiation.

79. The apparatus as set forth in claim 69, further comprising means to dispose a chemical agent to make said skin more or less transparent.

80. A method of providing one or more dermatological effects to a targeted portion of a human skin, comprising the step of:
providing a plurality of light source and optical pathway connections, wherein each light source in said plurality of light source and optical pathway connections is capable of delivering a light beam through its connected optical pathway to said targeted portion of a human skin;
providing a control means to select and control said light sources to deliver one or more light beams in a pattern; and
providing a focusing means to focus the power of said delivered light beams to tissue up to 1.5 mm underneath said targeted portion of said human skin.

81. A method of providing one or more dermatological effects to a targeted portion of a human skin, comprising the step of:
providing an optical delivery means, wherein said optical delivery means comprises an array of light source and optical pathways connections and wherein each light source in said optical delivery means is capable of delivering a light beam through its connected optical pathway to said targeted portion of a human skin, wherein said optical delivery means delivers one or more of said light beams in a pattern, wherein said one or more of said light sources comprises light beam parameters that results in dermatological effects; and
providing a focusing means, wherein said focusing means focuses the power of said delivered light beams to tissue up to 1.5 mm underneath said targeted portion of said human skin and wherein said focusing means deforms said targeted portion of said human skin.

82. A method of providing one or more dermatological effects to a targeted portion of a human skin, comprising the step of:
providing an optical delivery means, wherein said optical delivery means comprises an array of light source and optical pathways connections and wherein each laser in said optical delivery means is capable of delivering a light beam through its connected optical pathway to a targeted portion of a human skin, wherein said optical delivery means delivers one or more of said light beams in a pattern, wherein said one or more of said light sources comprises light beam parameters that results in dermatological effects;
providing a skin deformation means to deform said targeted portion of said human skin, wherein said skin deformation means comprises a vacuum means to apply a vacuum at said targeted portion of said human skin; and
providing a focusing means, wherein said focusing means focuses the power of said delivered light beams to tissue up to 1.5 mm underneath said targeted portion of said human skin.

83. A method of providing one or more dermatological effects to a targeted portion of a human skin, comprising the step of:
providing an optical delivery means, wherein said optical delivery means comprises an array of light source and optical pathways connections and wherein each laser in said optical delivery means is capable of delivering a light beam through its connected optical pathway to a targeted portion of a human skin, wherein said optical delivery means delivers one or more of said light beams in a pattern, wherein said one or more of said light sources comprises light beam parameters that results in dermatological effects;
providing a focusing means, wherein said focusing means focuses the power of said delivered light beams to tissue up to 1.5 mm underneath said targeted portion of said human skin, wherein said focusing means is also used as a skin deformation means to deform said targeted portion of said human skin.

84. A method of providing one or more dermatological effects to a targeted portion of a human skin, comprising the step of:
providing an optical delivery means, wherein said optical delivery means comprises an array of light source and optical pathways connections and wherein each light source in said optical delivery means is capable of delivering a light beam through its connected optical pathway to a targeted portion of a human skin, wherein said optical delivery means delivers one or more of said light beams in a pattern, wherein said one or more of said light sources comprises light beam parameters that results in dermatological effects;
providing a skin deformation means to deform said targeted portion of said human skin;
providing a focusing means, wherein said focusing means focuses the power of said delivered light beams to tissue up to 1.5 mm underneath said targeted portion of said human skin; and
providing a recording means to record said reflected radiation from said targeted portion of said human skin.

85. A method of providing one or more dermatological effects to a targeted portion of a human skin, comprising the step of:
an optical delivery means, wherein said optical delivery means comprises an array of light source and optical pathways connections and wherein each light source in said optical delivery means is capable of delivering a light beam through its connected optical pathway to a targeted portion of a human skin, wherein said optical delivery means delivers one or more of said light beams in a pattern, wherein said one or more of said light sources comprises light beam parameters that results in dermatological effects;
a skin deformation means to deform said targeted portion of said human skin;
a focusing means, wherein said focusing means focuses the power of said delivered light beams to tissue up to 1.5 mm underneath said targeted portion of said human skin; and
a viewing means to enable a user to view said targeted portion of said human skin, wherein said viewing means comprises a coating to protect said user's eyes from reflections of said light beams.

86. A dermatological laser system comprising:
a plurality of laser light sources;
a plurality of optical pathways coupled respectively to the plurality of laser light sources;
a control system coupled electronically to the plurality of laser light sources for individually controlling the operation of each of the plurality of laser light sources; and
at least one lens for focusing one or more beams of energy generated by the laser light sources and carried by an optical pathway at a desired tissue location within an epi-dermis, dermis, or hypo-dermis layer of human skin.

87. The dermatological laser apparatus of claim 86 wherein the control system comprises a microprocessor and related memory.

88. The dermatological laser apparatus of claim 87 further comprising a program that, when executed by the microprocessor, causes the microprocessor to selectively activate one or more of the plurality of laser light sources and thereby causes the dermatological laser apparatus to generate a selected pattern of beams that are delivered to the desired location.

89. A dermatological laser apparatus comprising:
a plurality of laser light sources configured to generate a plurality of beams having selected wavelengths between 400 nm and 5 μm;
an optical delivery system coupled to the plurality of laser light sources and including a set of optical elements that are configured to deliver and focus the beams generated by the plurality of laser light sources at a selected region of tissue on or within an area of skin of a patient; and
a control system coupled electronically to the plurality of laser light sources.

90. The dermatological laser apparatus of claim 89, wherein the control system and optical delivery system are configured such that a beam output delivered by the optical delivery system comprises a blended beam having multiple frequency components defined by said selected wavelengths.

91. The dermatological laser apparatus of claim 89, wherein the control system and optical delivery system are configured such that an output delivered by the optical delivery system comprises a pattern of beams having a plurality of differing wavelengths.

92. The dermatological laser apparatus of claim 91, wherein the output delivered by the optical delivery system comprises a pattern of beams having microscopic spot sizes at said selected region of tissue.
